(19) United States
(12) Patent Application Publication
(10) Pub. No.: US 2019/0167231 A1
(43) Pub. Date: Jun. 6, 2019

(54) SYSTEM AND METHOD FOR ULTRASONIC TISSUE SCREENING

(71) Applicant: SonoCine, Inc., Reno, NV (US)

(72) Inventor: Kevin M. Kelly, Venice, CA (US)

(21) Appl. No.: 16/205,306

(22) Filed: Nov. 30, 2018

Related U.S. Application Data
(60) Provisional application No. 62/593,730, filed on Dec. 1, 2017.

(51) Int. Cl. A61B 8/00; A61B 8/08; G06T 3/40; A61B 8/14

(52) U.S. Cl. CPC A61B 8/4461; A61B 8/4209; A61B 8/5223; A61B 8/54; A61B 8/0825; A61B 8/5246; A61B 8/085; A61B 8/463; A61B 8/145; A61B 8/5207; G06T 3/40

(57) ABSTRACT

A system for screening cellular tissue, including: an ultrasound scanning device including an ultrasound probe; a carrier device including a probe mount and configured to move the probe mount over the cellular tissue, the ultrasound probe being coupled to the probe mount to generate cross-sectional images of the cellular tissue; and a programmable device operably coupled to the carrier device and to the ultrasound scanning device, the programmable device configured to move the probe mount over the cellular tissue and to control the ultrasound scanning device to generate the cross-sectional images of the cellular tissue as the probe mount is moved, each cross-sectional image being approximately parallel to and having an image spacing with respect to one or more adjacent cross-sectional images, wherein the programmable device is configured to set the image spacing according to a predetermined ratio between a target minimum abnormality size and an effective number of images.

### CROSS REFERENCE TO RELATED APPLICATIONS

Priority is claimed to U.S. Provisional Application No. 62/593,730, filed Dec. 1, 2017, the disclosure of which is incorporated herein by reference in its entirety.

### FIELD OF THE INVENTION

The field of the present invention relates to systems and methods for screening cellular tissue for abnormalities using ultrasound.

### BACKGROUND OF THE INVENTION

Screening of breast tissue for potential abnormalities is very different than diagnosis of breast tissue for a cancerous abnormality. Screening breast tissue evaluates the entirety of the breasts for potential abnormalities quickly. It is used to evaluate women who have a low chance of having breast cancer at that moment and no focal finding of breast cancer. Breast tissue diagnosis is the evaluation of a region of a breast that may be abnormal based on clinical findings or abnormal screening findings by any one of a number of modalities.

Breast screening can be performed with many different modalities: physical examination, mammography, MRI (Magnetic Resonance Imaging), MBI (Molecular Breast Imaging), and ultrasound scanning, among others. Ultrasound screening can be done in three ways: handheld scanning, 3-D scanning, and AWBUS (Automated Whole breast Ultrasound Scanning). All three attempt to cover the entirety of both breasts.

U.S. Pat. No. 6,932,768, incorporated as Appendix A hereto, discloses a system and method for screening cellular tissue using ultrasound, which is a solution for performing an AWBUS procedure. The system and method are particularly useful for screening breast tissue. The method includes acquiring a series of evenly spaced images of the tissue using an ultrasound scanning apparatus, and then subsequently enabling the acquired images to be viewed sequentially in a cinematic fashion so that potential abnormalities can be identified by attending medical personnel. The manner in which the images are sequentially viewed is controlled by the user, such that the user can control how quickly the sequential images are viewed and whether the images are viewed in a forward direction or a reverse direction.

It is therefore desirable to enable visualization of potential abnormalities at about 3 mm or larger with greater reliability, and minimizing the opportunity for human error, while also reducing the amount of time for the screening and discomfort to the individual undergoing the screening. As has been recently discovered, ultrasound systems and processes can be better controlled to improve screening for potential abnormalities, even small-sized ones, which in turn can increase the detection of potential abnormalities and help improve overall health, particularly where breast cancer is concerned.

### SUMMARY OF THE INVENTION

The present invention is directed toward a system and method for ultrasonic tissue screening. In particular, in both the system and method the spacing between acquired images is reduced to improve detection of small potential abnormalities. The spacing between the acquired images can be controlled to select the smallest sized abnormality that can be easily identified by cinematic viewing of the acquired images. In addition, the spacing between the images can be controlled in connection with the playback frame rate in order to improve detection of potential abnormalities by the human eye.

In one separate aspect of the present invention, a system for screening cellular tissue includes: an ultrasound scanning device including an ultrasound probe; a carrier device including a probe mount, the carrier device configured to move the probe mount over the cellular tissue, the ultrasound probe being coupled to the probe mount to generate cross-sectional images of the cellular tissue; and a programmable device operably coupled to the carrier device and to the ultrasound scanning device, the programmable device configured to move the probe mount over the cellular tissue and to control the ultrasound scanning device to generate the cross-sectional images of the cellular tissue as the probe mount is moved, each cross-sectional image being approximately parallel to and having an image spacing with respect to one or more adjacent cross-sectional images, wherein the programmable device is configured to set the image spacing according to a predetermined ratio between a target minimum abnormality size and an effective number of images.

In another separate aspect of the present invention, a system for screening cellular tissue includes: an ultrasound scanning device including an ultrasound probe; a carrier device including a probe mount, the carrier device configured to move the probe mount over the cellular tissue, the ultrasound probe being coupled to the probe mount to generate cross-sectional images of the cellular tissue; a programmable device operably coupled to the carrier device and to the ultrasound scanning device, the programmable device configured to move the probe mount over the cellular tissue and to control the ultrasound scanning device to generate the cross-sectional images of the cellular tissue as the probe mount is moved, each cross-sectional image being approximately parallel to and having an image spacing with respect to one or more adjacent cross-sectional images, wherein the programmable device is configured to set the image spacing according to a predetermined ratio between a target minimum abnormality size and an effective number of images, the target minimum abnormality size being input by a user; and a display screen operably coupled to the programmable device, wherein the programmable device is configured to display the cross-sectional images in a cinematic presentation on the display screen, the cinematic presentation having a frame rate based on the effective number of images in the predetermined ratio.

In yet another separate aspect of the present invention, a method for screening cellular tissue includes: positioning an ultrasound probe over cellular tissue to be screened, the ultrasound probe being part of an ultrasound scanning device and being coupled to a probe mount of a carrier device; controlling, using a programmable device operably coupled to the carrier device, movement of the probe mount to move the ultrasound probe over the cellular tissue; and controlling, using the programmable device operably coupled to the ultrasound scanning device, the ultrasound probe while the probe mount is moving to generate cross-sectional images of the cellular tissue, each cross-sectional image being approximately parallel to and having an image spacing with respect to one or more adjacent cross-sectional images, wherein the programmable device sets the image spacing according to a predetermined ratio between a target minimum abnormality size and an effective number of images.

In still another separate aspect of the present invention, a system for screening cellular tissue includes: an ultrasound scanning device including an ultrasound probe; a carrier device including a probe mount, the carrier device configured to move the probe mount over the cellular tissue, the ultrasound probe being coupled to the probe mount to generate cross-sectional images of the cellular tissue, the cross-sectional images having an image depth; and a programmable device operably coupled to the carrier device and to the ultrasound scanning device, the programmable device configured to determine an image acquisition time for a sample cross-sectional image of the cellular tissue taken at the image depth, move the probe mount over the cellular tissue at a probe mount speed, and control the ultrasound scanning device to generate the cross-sectional images of the cellular tissue as the probe mount is moved, wherein the programmable device determines the probe mount speed using the image acquisition time.

In still another separate aspect of the present invention, a method for screening cellular tissue includes: positioning an ultrasound probe over cellular tissue to be screened, the ultrasound probe being part of an ultrasound scanning device and being coupled to a probe mount of a carrier device; determining, using a programmable device operably coupled to the carrier device, an image acquisition time for a sample cross-sectional image of the cellular tissue having an image depth; controlling, using a programmable device, movement of the probe mount to move the ultrasound probe over the cellular tissue at a probe mount speed; controlling, using the programmable device, the ultrasound probe while the probe mount is moving to generate cross-sectional images of the cellular tissue, the cross-sectional images having the image depth, wherein the programmable device determines the probe mount speed using the image acquisition time.

Accordingly, an improved system and method for ultrasound tissue screening are disclosed. Advantages of the improvements will be apparent from the drawings and the description herein.

### BRIEF DESCRIPTION OF THE DRAWINGS

The foregoing summary, as well as the following detailed description of the exemplary embodiments, will be better understood when read in conjunction with the appended drawings. It should be understood, however, that the invention is not limited to the precise arrangements and instrumentalities shown in the following figures:

FIG. 1 schematically illustrates a system for screening cellular tissue.

FIG. 2 is a plan view of an integrated patient platform and carrier device.

FIG. 3 is a side view of the integrated patient platform and carrier device of FIG. 2.

FIG. 4 is an end view of the integrated patient platform and carrier device of FIG. 2.

FIG. 5 is a flow chart illustrating a method for screening cellular tissue in accordance with a first embodiment.

FIG. 6A is a schematic diagram showing a plurality of scan rows of ultrasound images made of a human breast.

FIG. 6B is a schematic diagram showing a single ultrasound image within tissue.

FIG. 7 is a schematic diagram showing a plurality of ultrasound images in relation to an abnormality having a target minimum abnormality size.

FIG. 8 is a flow chart illustrating a method for screening cellular tissue in accordance with a second embodiment.

FIG. 9 is a flow chart illustrating a method for screening cellular tissue in accordance with a third embodiment.

### DETAILED DESCRIPTION OF THE INVENTION

As used throughout, ranges are used as shorthand for describing each and every value that is within the range, and each range includes the stated end values for the respective range. Any value within the range can be selected as the terminus of the range. In addition, all references cited herein are hereby incorporated by referenced in their entireties. In the event of a conflict in a definition in the present disclosure and that of a cited reference, the present disclosure controls.

Features of the present invention may be implemented in software, hardware, firmware, or combinations thereof. The programmable processes described herein are not limited to any particular embodiment, and may be implemented in an operating system, application program, foreground or background processes, driver, or any combination thereof. The computer programmable processes may be executed on a single processor or on or across multiple processors.

Processors described herein may be any central processing unit (CPU), microprocessor, micro-controller, computational, or programmable device or circuit configured for executing computer program instructions (e.g. code). Various processors may be embodied in computer and/or server hardware and/or computing device of any suitable type (e.g. desktop, laptop, notebook, tablet, cellular phone, smart phone, PDA, etc.) and may include all the usual ancillary components necessary to form a functional data processing device including without limitation a bus, software and data storage such as volatile and non-volatile memory, input/output devices, a display screen, graphical user interfaces (GUIs), removable data storage, and wired and/or wireless communication interface devices including Wi-Fi, Bluetooth, LAN, etc.

Processes described herein may be implemented using computer-executable instructions or programs (e.g. software or code), and data described herein may be programmed into and tangibly embodied in a non-transitory computer-readable medium that is accessible to and retrievable by a respective processor as described herein which configures and directs the processor to perform the desired functions and processes by executing the instructions encoded in the medium. A device embodying a programmable processor configured to execute such non-transitory computer-executable instructions or programs is referred to hereinafter as a “programmable device”, or just a “device” for short, and multiple programmable devices in mutual communication is referred to as a “programmable system”.

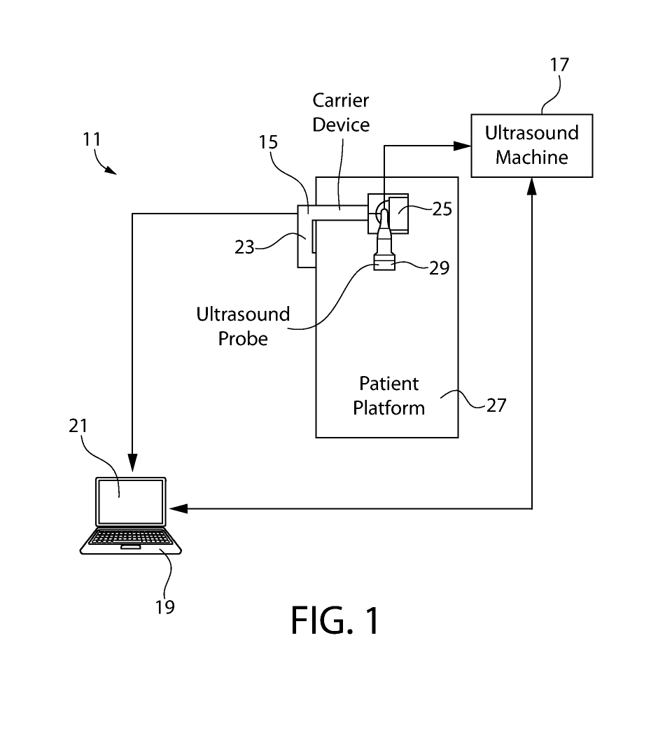

An ultrasound screening system 11 is shown in FIG. 1. The ultrasound screening system 11 includes a carrier device 15, an ultrasound scanning device 17, and a programmable device 19, all positioned around a patient platform 27. The programmable device 19 is operably coupled to the carrier device 15 and the ultrasound scanning device 17. The programmable device 19 may be any suitable type of programmable device, such as desktop, laptop, notebook, tablet, cellular phone, smart phone, PDA, FPGA computer, system on a chip, and the like. The programmable device 19 includes a processor (not shown) and a display screen 21 for displaying ultrasound images to the user. In certain embodiments, the display screen 21 may be separate and distinct from the programmable device 19 and still remain operably coupled to the programmable device 19. In such embodiments, the separate display screen 21 may still be used for displaying the ultrasound images. In still other embodiments, the display screen 21 may be incorporated as part of a second programmable device communicably coupled to the programmable device 19.

The carrier device 15 includes a support member 23 and a probe mount 25, and the support member 23 supports the probe mount 25 above the patient platform 27. In the embodiment shown, the support member 23 is coupled to the patient platform 27, and the patient platform 27 serves to steady the patient and provide a base for the carrier device 15. The patient platform 27 may be any appropriate surface for a patient to recline in a supine position, such as an examination table, a bed, a gurney, or the like. The support member 23 is capable of translational movement to laterally move the probe mount 25 over the patient platform 27, and thus also over the patient reposed on the platform and the tissue to be scanned. The programmable device 19 is programmed to control movement of the support member 23 for purposes of generating ultrasound images, as described below.

In certain other embodiments, the support member 23 can be either on a parallel track arrangement (one sided or multi-sided), or be configured as an articulating arm or some other contrivance, located over, underneath or adjacent to the patient (with or without the use of a patient platform) positioned either upright or prone. The carrier device 15 need not be supported by being coupled to the patient platform, but could be independently suspended from the ceiling, wall, or floor. The ultrasound probe may be supported and propelled by the carrier device 15 by any means (manually, mechanically, electrically, hydraulically, pneumatically or by any other means, with or without control feedback), or any combination of methods. These methods, singularly or combined may be utilized to control the ultrasound probe in the x-, y-, and z-axes. Gravity may also be employed to provide the requisite pressure of the ultrasound probe on the patient, or assist in the propulsion of the ultrasound probe across the cellular tissue. The ultrasound probe may be designed as a permanent or removable component of the carrier device 15.

The ultrasound scanning device 17 may be a standard medical ultrasound device, which is commercially available and includes an ultrasound probe 29. The ultrasound probe 29 is coupled to the probe mount 25 so that ultrasound energy is directed into the patient for generating the ultrasound images during the scan process. The ultrasound images may be generated by the ultrasound scanning device 17 using a scan mode that is common to standard medical ultrasound devices, and thus is not described herein in detail. The programmable device 19 is programmed to control the ultrasound scanning device 17 during the scan process as the support member 23 is moved above the tissue.

During the scan process, the programmable device 19 synchronizes control of both movement of the support member 23 and generation of the ultrasound images by the ultrasound scanning device 17 to acquire the ultrasound images as a plurality of approximately parallel cross-sectional ultrasound images of the tissue. Depending on the size of the scan head of the ultrasound probe 29, a full scan of the tissue may be obtained in several rows of cross-sectional ultrasound images. The programmable device 19 may then store the ultrasound images in a memory, and then display the ultrasound images in a cinematic presentation using the display screen 21.

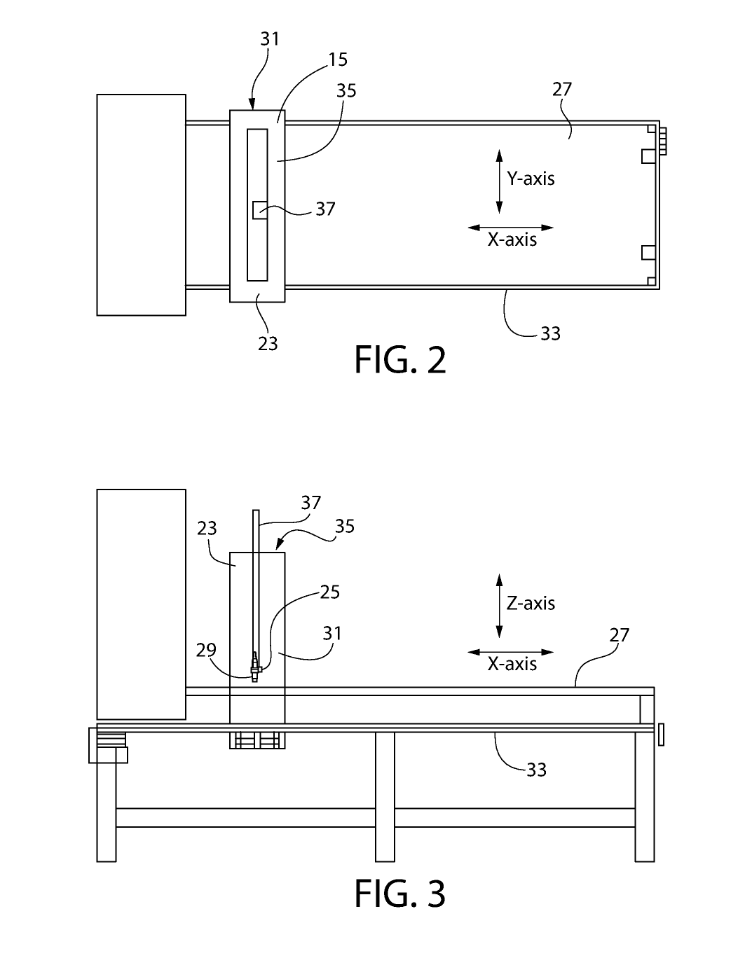

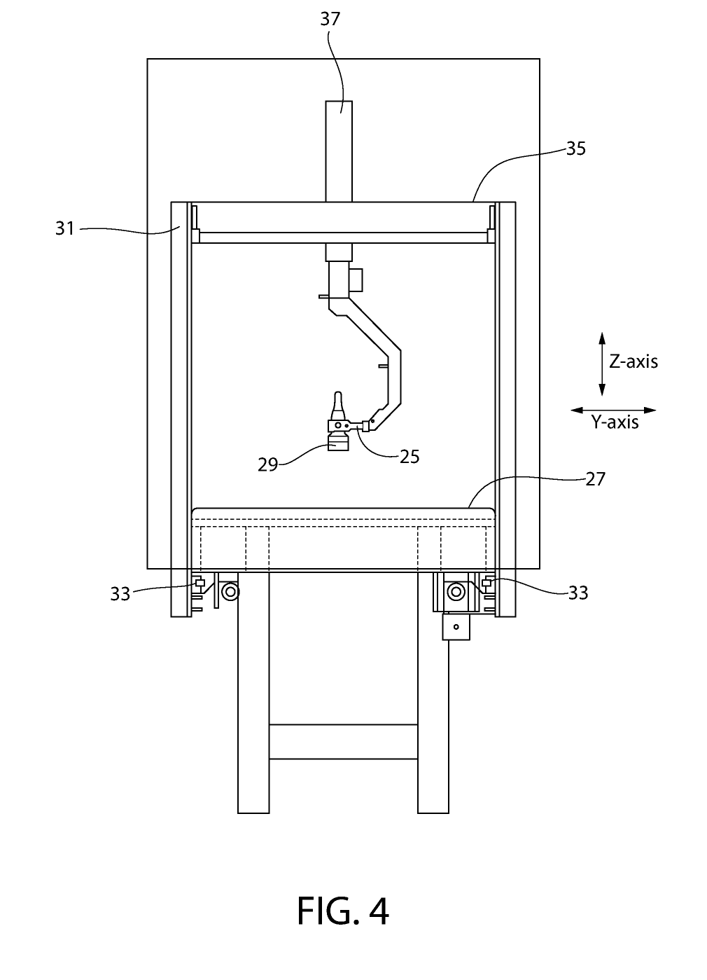

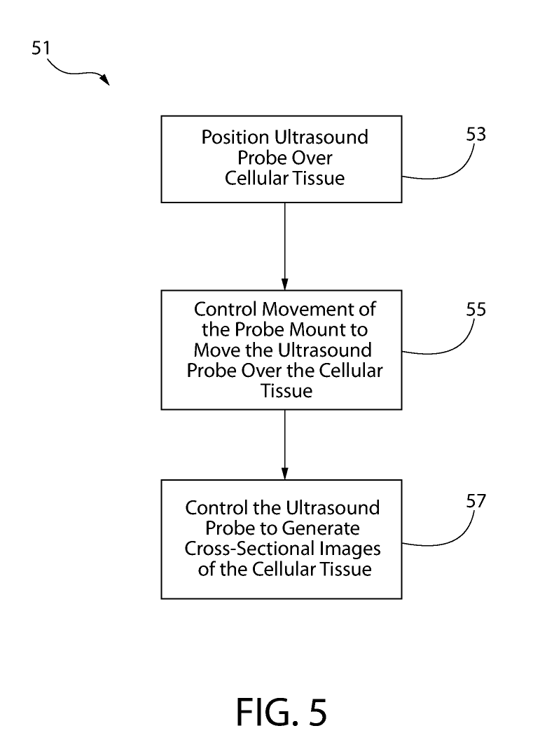

FIGS. 2-4 illustrate an embodiment of the carrier device 15 in greater detail. Although the carrier device 15 shows an exemplary device that may be used to move the ultrasound probe over the cellular tissue, it will be recognized that other mechanisms may be used to move the ultrasound probe mount. For example, in certain embodiments, the ultrasound probe may be moved using an articulating arm that enables positioning of the probe in an X-Y plane above the cellular tissue.

As shown, the carrier device 15 includes the support member 23 for coupling the carrier device 15 to the patient platform 27. The patient platform 27 steadies the patient during the exam and acts as a base for the carrier device 15. The support member 23 includes two parallel vertical members 31 coupled to rails 33 beneath the patient platform 27 and a horizontal member 35 that is coupled to the tops of the two vertical members 31. The rails 33 allow the support member 23 to move along the length of the patient platform 27, and this direction is defined herein as the x-axis. The probe mount 25 includes a vertical support 37 movably coupled to the horizontal member 35 between the two vertical members 31. The vertical support 37 is movable along the length of the horizontal member 35, and this direction is defined herein as the y-axis. The vertical support 37 also enables movement of the ultrasound probe 29 toward and away from the patient platform 27, and this direction is defined herein as the z-axis. With the carrier device 15 providing movement along three orthogonal axes, the ultrasound probe 29 may be held at a fixed angle with respect to the cellular tissue and positioned as needed to generate the ultrasound images in the manner described herein. In certain embodiments, the probe mount 25 may be articulated to hold the ultrasound probe 29 at any desired angle relative to the cellular tissue by rotating about the x- and y-axes. In such embodiments, the angle of the ultrasound probe 29 with respect to the cellular tissue may be dynamically changed during the scan process to keep the scan head of the ultrasound probe 29 perpendicular to the patient's skin (or any other preferred orientation).

The carriage 31 is propelled along the x-axis of the patient platform 27 during the scan process by one or more motors controlled by the programmable device 19. The probe mount 25 is moved along the y-axis during the scan process by one or more motors controlled by the programmable device 19. The probe mount 25 is also moved along the z-axis during the scan process by one or more motors controlled by the programmable device 19. In certain embodiments, microprocessors that are separate from the programmable device 19 may be used to move the probe mount along any one or more of the axes.

During the scan process, the probe mount 25 moves along the z-axis to maintain consistent contact between the ultrasound probe 29 and the patient's skin (the surface of the cellular tissue). In certain embodiments, the z-axis of the probe mount 25 is controlled to maintain a constant pressure of the ultrasound probe 29 on the patient's skin. In such embodiments, it may be desirable to control this constant pressure in accordance with a user-selected preset value. This pressure may monitored during the scan, and an override function may be programmed to move the probe mount 25 up and away from the patient, i.e., movement along the z-axis) if a maximum pressure level is detected or exceeded. When the scan process is complete, the probe mount 25 is controlled to move upward along the z-axis, away from the patient, so that the patient may leave the patient platform 27.

All the data related to the position (and angle, if the angle of the probe mount 27 is controlled) of the ultrasound probe 29 are provided to the viewing program to allow the ultrasound images to be correlated with their corresponding location on the patient. The position data also allows the programmable device 19 to compensate for the overlapping of, or gaps between images, that may be present in the ultrasound image set resulting from the scan process.

During the screening process, the speed of the probe mount 25 (referred to herein as the “probe mount speed”) is precisely controlled by the programmable device 19. The ultrasound scanning device 17 is controlled by the programmable device 19 so that the ultrasound probe 29 is sequentially activated as it moves across the cellular tissue. The sequential activation of the ultrasound probe 29 is controlled in conjunction with the probe mount speed to achieve the desired spacing between adjacent ultrasound images. By controlling the probe mount speed of the ultrasound scanning device 17, ultrasound images may be generated having uniform spacing between adjacent images. In certain embodiments, the probe mount speed may be controlled to be a constant speed, thus facilitating control of the ultrasound scanning device 17 for generation of the ultrasound images. This uniform spacing of the scan process may be advantageously used when the ultrasound images are viewed in a cinematic presentation as part of the overall screening process. The manner in which the image spacing and the cinematic presentation are controlled by the programmable device 19 are discussed in greater detail below as part of the cellular tissue screening process.

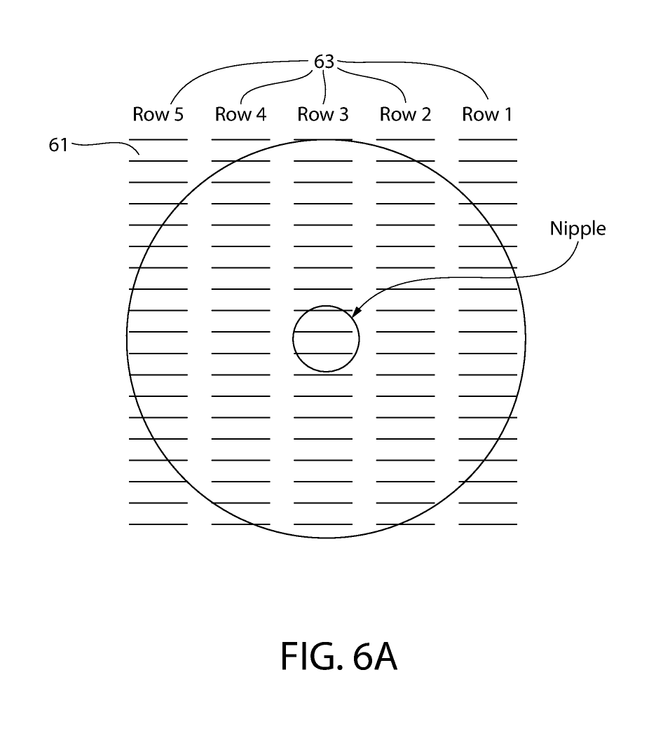

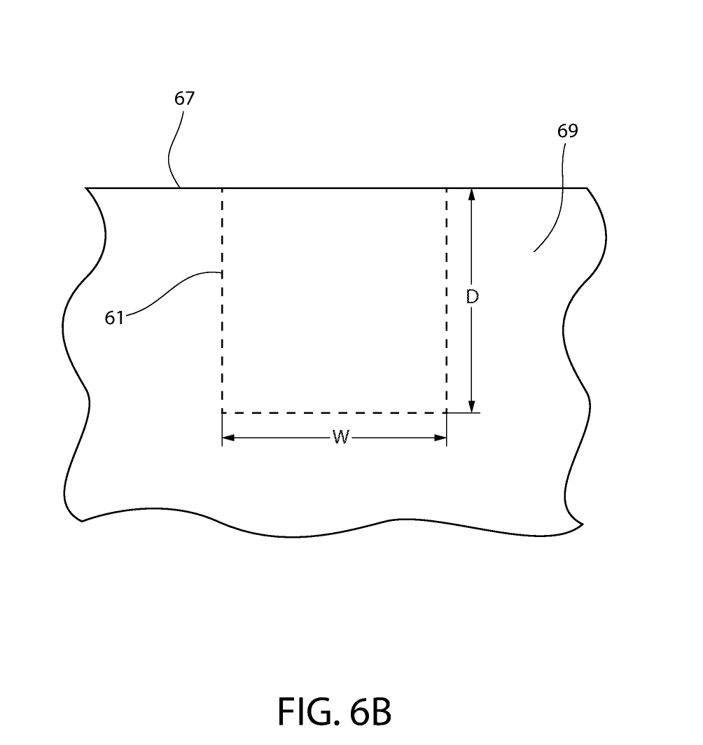

The flowchart of FIG. 5 shows a screening process 51 for generating ultrasound images of cellular tissue using the ultrasound screening system of FIG. 1. For purposes of clarity, the cellular tissue is breast tissue. However, it will be understood that the cellular tissue may be any type of cellular tissue. The programmable device 19 may be programmed to carry out the steps of this screening process 51 where it is amenable to automation. The first step of the screening process 51 is to position 53 the ultrasound probe over the cellular tissue. In certain embodiments, the initial positioning of the ultrasound probe may require input from a user. Once the ultrasound probe is positioned 53, then movement of the probe mount is controlled 55 to move the ultrasound probe over the cellular tissue. While movement of the probe mount is being controlled 55, the ultrasound probe is simultaneously controlled 57 to generate cross-sectional (ultrasound) images of the cellular tissue. By controlling 55 the probe mount speed while simultaneously controlling 57 generation of the ultrasound images, the programmable device 19 may control spacing between adjacent images with a high degree of accuracy. In particular, the programmable device 19 may be programmed to set the image spacing based on a predetermined ratio between a target minimum abnormality size and the image spacing. The programmable device 19 may also be programmed to display the generated images in a cinematic presentation, with the frame rate of the cinematic presentation being based on the effective number of images in the predetermined ratio. These programmed features are described in greater detail below with respect to FIGS. 6-8.

As preparation for the screening process 51, the user, who may be a physician, scan technician, or other individual, determines the area of the breast that is to be scanned. In current practice, the width of the breast tissue scanned by the ultrasound probe is generally too large to capture the entire width of the tissue in a single scan image. As a result, several adjacent passes are performed to provide complete coverage. Each pass (referred to herein as a “scan row”) will have some overlap with the preceding pass to achieve full coverage and eliminate the potential for missing features at the fringes of the scan. Prior to each successive pass for each scan row, the probe mount 25 lifts away from the patient, moves along the y-axis across the breast tissue and along the x-axis to the top of the breast tissue to be in position for the next scan row, at which point the probe mount 25 is then lowered along the z-axis onto the patient again.

A scan row contains a plurality of individual ultrasound images typically about 200 to 300 or more per breast. FIG. 6A depicts how the ultrasound images 61 are arranged in a plurality of scan rows 63 on a typical breast scan, but for clarity, no overlap is shown. A scan row 63 can be thought of as a stack of photographic slides, each slide representing an individual ultrasound image 61, which is shown in FIG. 6B. Each ultrasound image 61 includes a width W, which is determined by the width of the ultrasound probe head, and extends a depth D below the tissue surface 67 to image the cellular tissue 69. The depth D of the ultrasound image is generally adjustable for most ultrasound scanning devices available on the market. For example, the depth D may be reduced when the cellular tissue to be imaged is shallow, and the depth D may be increased when the cellular tissue to be imaged extends more deeply into the body. In certain embodiments, the depth D may be set as user input to the ultrasound scanning device. In certain other embodiments, the depth D may be set as user input to the programmable device 19, and in such embodiments, the programmable device controls the ultrasound scanning device so that the ultrasound images are generated at the depth set by the user. As discussed in further detail below, the image acquisition time is dependent, at least in part, upon the depth D of the ultrasound image, with deeper images having a longer image acquisition time. In certain embodiments, the programmable device may control the probe mount speed based on the image acquisition time in order to generate ultrasound images with the desired image spacing and image depth.

In certain embodiments, the ultrasound images 61 are evenly spaced, and each ultrasound image 61 is substantially parallel to adjacent ultrasound images 61. This arrangement of the ultrasound images 61 may be accomplished by uniform motion of the probe 8 and uniform timing of ultrasound sound probe when generating the ultrasound images 61 in an individual scan row 63. In such embodiments, the angle of the ultrasound probe is not rotated about the x- and y-axes during the screening process 51.

In certain other embodiments, however, one or more of the ultrasound images 61 in a scan row 63 may deviate away from being substantially parallel with each other, with the spacing between individual frames being determined by the position of a fixed point on one of the ultrasound probe or the probe mount at the time each ultrasound image 61 is generated. In such embodiments, the deviation away from substantially parallel between two adjacent ultrasound images may be up to about 10°.

In embodiments in which the ultrasound images deviate away from substantially parallel, the angular position of the ultrasound probe may be dynamically adjusted during the screening process to follow the contours of the cellular tissue being scanned. In such embodiments, the tops of each ultrasound image may be evenly spaced with adjacent images, as the contours of the cellular tissue may be sufficiently gentle that adjacent ultrasound images will deviate from parallel by no more than a few degrees. Although adjacent ultrasound images within a single scan row may deviate from parallel only slightly, non-adjacent ultrasound images may become progressively less parallel as they are separated by an increasing number of other ultrasound images. Additionally, ultrasound images in different scan rows may not necessarily be parallel.

In certain embodiments of a full screening process, two breasts may be scanned in four segments, each segment including one-half of a breast. Each segment includes multiple scan rows 63, with the first scan row aligned at the center of the breast over the nipple and successive scan rows being progressively further from the nipple. In other embodiments, each breast may be scanned in one or more segments, with the scan rows progressing across the entire breast from lateral to medial, or vice-versa.

During the screening process 51, the image spacing is selected so that there is an effective number of ultrasound images with respect to the target minimum abnormality size, resulting in a predetermined ratio between the target minimum abnormality size and the effective number of images. As used herein, the target minimum abnormality size is a dimensional width of the smallest potential abnormality that is sought to be found through the screening process. Prior to movement of the ultrasound probe and generation of the ultrasound images, the user may select the image spacing by identifying the target minimum abnormality size that is sought to be found through the screening process 51. With the target minimum abnormality size identified by the user, the image spacing is determined by application of the predetermined ratio.

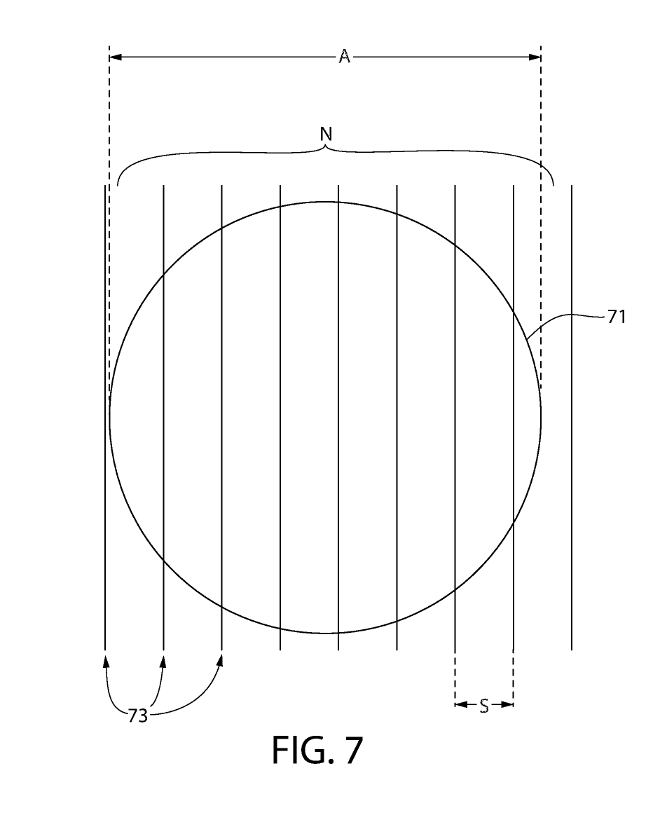

The predetermined ratio is illustrated in FIG. 7. An abnormality 71 having the target minimum abnormality size is shown superimposed over a plurality of ultrasound images 73. As shown, the target minimum abnormality size is A, and the effective number of images is N, where N may be a non-whole number, such that the predetermined ratio may be stated as A:N. The resulting image spacing, S, is then determined as A/N. In certain embodiments, the effective number of images is 7.5. In certain other embodiments, the effective number of images may be set from 6 up to 10, or even higher.

In screening processes in which the predetermined ratio between the target minimum abnormality size and the effective number of images is selected as A:7.5, by way of examples, when the target minimum abnormality size is 3 mm, the image spacing is selected as about 0.4 mm; when the target minimum abnormality size is 4 mm, the image spacing is selected as about 0.53 mm; when the target minimum abnormality size is 5 mm, the image spacing is selected as about 0.67 mm; when the target minimum abnormality size is 6 mm, the image spacing is selected as about 0.8 mm; when the target minimum abnormality size is 7 mm, the image spacing is selected as about 0.93 mm; when the target minimum abnormality size is 8 mm, the image spacing is selected as about 1.07 mm; when the target minimum abnormality size is 9 mm, the image spacing is selected as about 1.2 mm; and when the target minimum abnormality size is 10 mm, the image spacing is selected as about 1.33 mm.

Although different predetermined ratios may be used for any target minimum abnormality size, there is a tradeoff in the speed at which ultrasound images are generated based upon the selected ratio and the target minimum abnormality size because of the number images that must be generated at higher ratios when the target minimum abnormality size is set to 5 mm and smaller.

Comparing the screening process disclosed herein with existing cellular tissue screening modalities, the smallest potential abnormality that most current screening modalities can detect are inherent within the technology of the modality itself or based upon the type of tissue being screened. For example, most mammograms can detect a potential abnormality of about 10 mm, but only in breast tissue that is not very dense. By way of another example, AWBUS can detect a potential abnormality of about 5 mm in breast tissue that is dense or not dense. No existing scanning modality enables changing the smallest potential abnormality that is targeted by the respective modality.

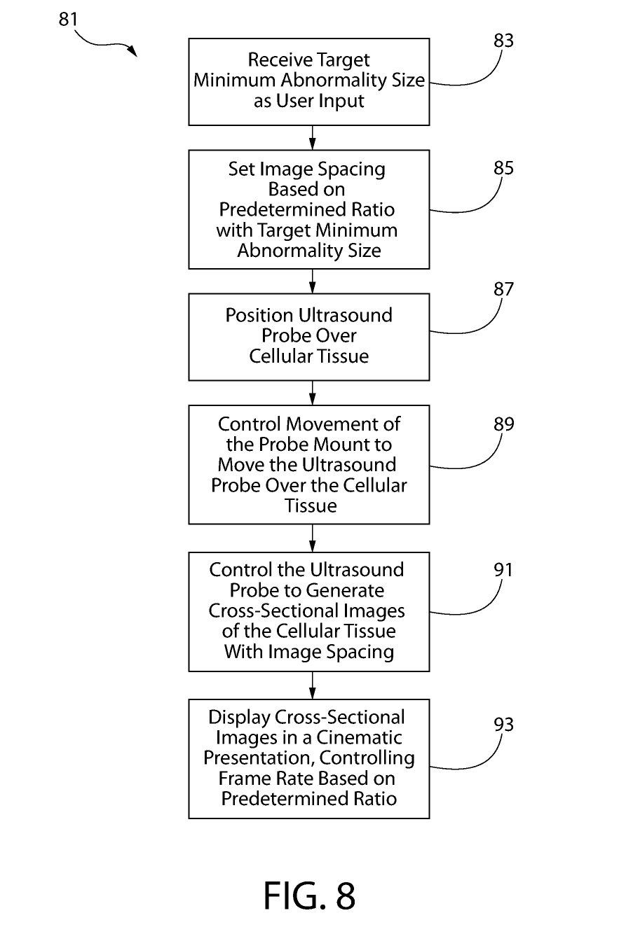

FIG. 8 is a flowchart illustrating a screening process 81 in which ultrasound images of cellular tissue are generated and the images are displayed in a cinematic presentation. The programmable device 19 may be programmed to carry out the steps of this screening process 81 where it is amenable to automation. The first step in this screening process 81 is to receive 83 the target minimum abnormality size as input from the user. The image spacing is then set 85 based on application of the predetermined ratio to the input target minimum abnormality size. Next, the ultrasound probe over is positioned 87 over the cellular tissue being screened. In certain embodiments, the initial positioning of the ultrasound probe may require input from a user. This input may be in the form of the user placing the probe mount at a starting position. Once the ultrasound probe is positioned 87, then movement of the probe mount is controlled 89 to move the ultrasound probe over the cellular tissue. While movement of the probe mount is being controlled 89, the ultrasound probe is simultaneously controlled 91 to generate cross-sectional ultrasound images of the cellular tissue. Once the cross-sectional images have been generated, then they are displayed 93 in a cinematic presentation in which the frame rate is controlled based on the effective number of frames in the predetermined ratio.

During display of the ultrasound images in the cinematic presentation, the image delivery, playback speed, image pixel resolution, display image size ratio to screen size, and image weighting (with dark border) can be optimized for delivery of the series of ultrasound images in the cinematic presentation to the macula of the eye. Control of the frame rate of the cinematic presentation, in combination with the effective number of images in the predetermined ratio, ensures that enough image slices are present to allow detection of potential abnormalities. With appropriate selection of the image spacing, potential abnormalities as small as 3 mm in size, or even smaller, may be detected. The optimized factors allow detection as the cinematic presentation presents each potential abnormality as a rapid change on the display screen so that areas of suspicion flash in and out of visual perception, thereby mimicking short bursts of motion. This mimicking of motion provides the cinematic presentation with the following advantages:
Unusual motion alerts a person and naturally draws attention.
A person responds to things that move “unexpectedly.”
The cinematic presentation results in the amount of image data generated being proportional to the smallest targeted size of potential abnormality (i.e., the target minimum abnormality size), as opposed the amount of image data generated being proportional to the size of the tissue being scanned as is done with typical ultrasound screening modalities.
It is designed to maximize visualization and detectability of potential abnormalities by presenting the generated ultrasound images in cinematic format in which the display frame rate is also selected to be proportional to the effective number of images in the predetermined ratio, as described below, thereby causing potential abnormalities to “pop out”.

Additional advantages may be obtained for the cinematic presentation through the environment of how the cinematic presentation is viewed. One such advantage may be realized by placing the cinematic presentation at a reading distance from the eye of the user in order to focus the image on the maculae of the user and eliminate the need for eye movement during review. In certain embodiments, the size of the images included in the cinematic presentation may be adjusted to take into account the viewing distance of the user. Another advantage may be realized by blocking extraneous light from entering the user's eyes by placing the cinematic presentation within wide black peripheral border. In alternative embodiments, a screen hood may also be used to further block extraneous light.

During the cinematic presentation, the frame rate at which the ultrasound images are displayed may be selected to be proportional to the effective number of images in the predetermined ratio, such that all of the effective number of images for any given target minimum abnormality size are displayed in about 0.25 seconds of the cinematic presentation. By way of examples, for a cinematic presentation in which the predetermined ratio is A:7.5, the frame rate of the cinematic presentation is set to 30 frames per second; for a cinematic presentation in which the predetermined ratio is A:6, the frame rate is set to 24 frames per second; for a cinematic presentation in which the predetermined ratio is A:8, the frame rate is set to 32 frames per second; for a cinematic presentation in which the predetermined ratio is A:9, the frame rate is set to 36 frames per second; and for a cinematic presentation in which the predetermined ratio is A:10, the frame rate is set to 40 frames per second. As will be evident, the greater the effective number of images in the predetermined ratio, the longer the image generation process takes, and the fewer the effective number of images in the predetermined ratio, the shorter the image generation process takes. The ratio of A:7.5, therefore, is intended to be a balance between the length of time required for image generation, the processing requirements for displaying the cinematic presentation, and the ease of identifying potential abnormalities using the cinematic presentation.

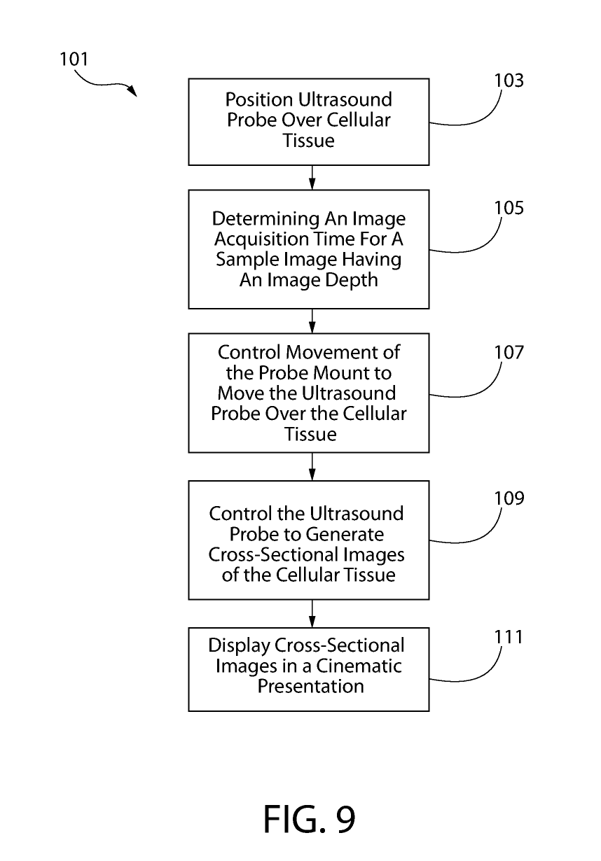

Turning to FIG. 9, a flowchart illustrating another screening process 101 in which ultrasound images of cellular tissue are generated and the images are displayed in a cinematic presentation. In this screening process 101, the programmable device uses the image acquisition time of the ultrasound scanning device to control the probe mount speed, and thereby enable the ultrasound images to be acquired at the desired image spacing. Different ultrasound scanning devices may have different image acquisition times, even for ultrasound images generated having the same depth. In addition, ultrasound images that are generated having different depths will also have different image acquisition times. In the screening process 101, the programmable device uses image acquisition time of the ultrasound device, whether the image acquisition time results from a default setting or from a change in the image depth, to adjust the probe mount speed and obtain the desired image spacing for the generated ultrasound images. Maintaining the image spacing can greatly assist in identifying potential abnormalities by ensuring that a full and complete scan of the cellular tissue is performed. And, when the generated ultrasound images are displayed in a cinematic presentation, such a full and complete scan of the cellular tissue aids in identifying all potential abnormalities that are the target minimum abnormality size or larger.

In certain embodiments, the image depth of the ultrasound images may be set by the user, and changes to the image depth result in changes to the image acquisition time for each ultrasound image. Particularly, an increase in image depth will increase the image acquisition time for each ultrasound image, and a decrease in image depth will decrease the image acquisition time for each ultrasound image. For any change in the image depth, ΔD, whether an increase or decrease, the distance traveled by the ultrasound energy within the cellular tissue is increased or decreased, respectively, by twice the change in depth 2ΔD when generating an ultrasound image. By way of example, since the speed of ultrasound waves through soft tissue is approximately 1.54 mm/μs, each 5 mm change in image depth will result in an increase or decrease of about 6.5 μs in the image acquisition time for each ultrasound image. Any change in image acquisition time can impact a screening process which seeks to maintain a consistent image spacing between adjacent ultrasound images. For example, during use of the system of FIG. 1, if the probe mount speed is too fast and the image acquisition time is too long, then smaller image spacing may not be achievable. In such instances, the probe mount speed would need to be reduced to accommodate the longer image acquisition time and achieve the desired image spacing. Conversely, if the probe mount speed is too slow, then the process of generating the entire set of ultrasound images, such as the multiple scan rows shown in FIG. 6A, may take longer than necessary for the comfort of the patient. Adjusting, and even optimizing, the probe mount speed to take into account the image acquisition time is therefore highly desirable.

As shown in FIG. 9, the image depth may be automatically adjusted by the programmable device in order to control movement of the probe mount, particularly the probe mount speed, based on the image acquisition time. The first step in this screening process 101 is to position 103 the ultrasound probe over the cellular tissue being screened. In certain embodiments, the initial positioning of the ultrasound probe may require input from a user. This input may be in the form of the user placing the probe mount at a starting position. Once the ultrasound probe is positioned, the programmable device determines 105 the image acquisition time for a sample ultrasound image having an image depth. In certain embodiments of the screening process 101, the image depth of the sample ultrasound image may be the default depth of the ultrasound scanning device coupled to the programmable device. In certain other embodiments of the screening process 101, the image depth of the sample ultrasound image may be an image depth set according to user input into one of the programmable device or the ultrasound scanning device. With the image acquisition time known, the probe mount speed may be adjusted in the step of controlling movement 107 of the probe mount to move the ultrasound probe over the cellular tissue. While movement of the probe mount is being controlled 107, the ultrasound probe is simultaneously controlled 109 to generate cross-sectional ultrasound images of the cellular tissue. As part of this controlling movement 107 step, the probe mount speed may be decreased if needed to ensure that the full depth of the generated ultrasound images is properly captured, or the probe mount speed may be increased to reduce the duration of the overall screening process 101, thereby increasing the comfort of the patient. In certain embodiments, the probe mount speed may be set to a constant speed within a scan row in order to facilitate movement of the probe mount and generation of the ultrasound images. Once the cross-sectional images have been generated, then they are displayed 111 in a cinematic presentation. In certain embodiments, all ultrasound images generated during the screening process 101 have the same image depth.

The improvements in ultrasound screening described above can be used for all types of breast tissue equally, including those with scar tissue and implants, including circumstances in which:

a. the breasts are almost entirely fatty;

b. there are scattered areas of fibroglandular density;

c. the breasts are heterogeneously dense, which may obscure small masses;

d. the breasts are extremely dense, which lowers the sensitivity of mammography.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. A system for screening cellular tissue, the system comprising:
an ultrasound scanning device including an ultrasound probe;
a carrier device including a probe mount, the carrier device configured to move the probe mount over the cellular tissue, the ultrasound probe being coupled to the probe mount to generate cross-sectional images of the cellular tissue; and
a programmable device operably coupled to the carrier device and to the ultrasound scanning device, the programmable device configured to move the probe mount over the cellular tissue and to control the ultrasound scanning device to generate the cross-sectional images of the cellular tissue as the probe mount is moved, each cross-sectional image being approximately parallel to and having an image spacing with respect to one or more adjacent cross-sectional images,
wherein the programmable device is configured to set the image spacing according to a predetermined ratio between a target minimum abnormality size and an effective number of images.

2. The system of claim 1, wherein the target minimum abnormality size is input by a user.

3. The system of claim 1, wherein the target minimum abnormality size ranges between 3 mm and 10 mm.

4. The system of claim 1, wherein the predetermined ratio between the target minimum abnormality size and the effective number of images is 1:7.5.

5. The system of claim 1, wherein the predetermined ratio between the target minimum abnormality size and the effective number of images ranges between 1:6 and 1:10.

6. The system of claim 1, further comprising:
a display screen operably coupled to the programmable device, wherein the programmable device is configured to display the cross-sectional images in a cinematic presentation on the display screen, the cinematic presentation having a frame rate based on the effective number of images in the predetermined ratio.

7. The system of claim 6, wherein the programmable device is configured to set the frame rate to a whole number multiple of the effective number of images in the predetermined ratio.

8. (canceled)

9. The system of claim 6, wherein the programmable device is configured to adjust a size of the cross-sectional images in the cinematic presentation in accordance with a viewing distance of a user from the display screen.

10. A system for screening cellular tissue, the system comprising:
an ultrasound scanning device including an ultrasound probe;
a carrier device including a probe mount, the carrier device configured to move the probe mount over the cellular tissue, the ultrasound probe being coupled to the probe mount to generate cross-sectional images of the cellular tissue;
a programmable device operably coupled to the carrier device and to the ultrasound scanning device, the programmable device configured to move the probe mount over the cellular tissue and to control the ultrasound scanning device to generate the cross-sectional images of the cellular tissue as the probe mount is moved, each cross-sectional image being approximately parallel to and having an image spacing with respect to one or more adjacent cross-sectional images, wherein the programmable device is configured to set the image spacing according to a predetermined ratio between a target minimum abnormality size and an effective number of images, the target minimum abnormality size being input by a user; and
a display screen operably coupled to the programmable device, wherein the programmable device is configured to display the cross-sectional images in a cinematic presentation on the display screen, the cinematic presentation having a frame rate based on the effective number of images in the predetermined ratio.

11. The system of claim 10, wherein the target minimum abnormality size ranges between 3 mm and 10 mm.

12. The system of claim 10, wherein the predetermined ratio between the target minimum abnormality size and the effective number of images is 1:7.5.

13. The system of claim 10, wherein the predetermined ratio between the target minimum abnormality size and the effective number of images ranges between 1:6 and 1:10.

14. The system of claim 10, wherein the programmable device is configured to set the frame rate to a whole number multiple of the effective number of images in the predetermined ratio.

15. (canceled)

16. The system of claim 10, wherein the programmable device is configured to adjust a size of the cross-sectional images in the cinematic presentation in accordance with a viewing distance of a user from the display screen.

17. A method for screening cellular tissue, the method comprising:
positioning an ultrasound probe over cellular tissue to be screened, the ultrasound probe being part of an ultrasound scanning device and being coupled to a probe mount of a carrier device;
controlling, using a programmable device operably coupled to the carrier device, movement of the probe mount to move the ultrasound probe over the cellular tissue;
controlling, using the programmable device operably coupled to the ultrasound scanning device, the ultrasound probe while the probe mount is moving to generate cross-sectional images of the cellular tissue, each cross-sectional image being approximately parallel to and having an image spacing with respect to one or more adjacent cross-sectional images, wherein the programmable device sets the image spacing according to a predetermined ratio between a target minimum abnormality size and an effective number of images.

18. The method of claim 17, further comprising receiving, by the programmable device, the target minimum abnormality size input by a user.

19. The method of claim 17, wherein the target minimum abnormality size ranges between 3 mm and 10 mm.

20. The method of claim 17, wherein the predetermined ratio between the target minimum abnormality size and the effective number of images is 1:7.5.

21. The method of claim 17, wherein the predetermined ratio between the target minimum abnormality size and the effective number of images ranges between 1:6 and 1:10.

22. The method of claim 17, further comprising:
displaying the cross-sectional images in a cinematic presentation on a display screen, the cinematic presentation having a frame rate based on the effective number of images in the predetermined ratio.

23. The method of claim 22, further comprising setting, using the programmable device, the frame rate to a whole number multiple of the effective number of images in the predetermined ratio.

24. (canceled)

25. The method of claim 22, further comprising setting, using the programmable device, a size of the cross-sectional images in the cinematic presentation in accordance with a viewing distance of a user from the display screen.

26.-40. (canceled)
